(19) United States
(12) Patent Application Publication
(10) Pub. No.: US 2024/0148497 A1
(43) Pub. Date: May 9, 2024

(54) PROSTHETIC HEART VALVE FRAMES WITH RADIALLY OFFSET COMMISSURE PORTIONS

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Ziv Yohanan, Kfar Hahoresh (IL); Anatoly Dvorsky, Haifa (IL); Eran Grosu, Kfar-Saba (IL)

(21) Appl. No.: 18/408,174

(22) Filed: Jan. 9, 2024

Related U.S. Application Data
(63) Continuation of application No. PCT/US2022/037864, filed on Jul. 21, 2022.
(60) Provisional application No. 63/226,328, filed on Jul. 28, 2021.

(51) Int. Cl. A61F 2/24

(52) U.S. Cl. CPC A61F 2/2418; A61F 2/2433; A61F 2/2436; A61F 2220/0025

(57) ABSTRACT

Described herein are annular frames for prosthetic heart valves that include commissure portions that are radially offset and/or are radially flexible relative to the rest of the frame. The commissure portions can extend radially outwardly from the rest of the frame such that they are positioned at a greater radius than the adjacent struts of the frame. The commissure portions can be cantilevered from the frame, such that only one axial end of the commissure portion is attached to the rest of the frame, allowing the commissure portions to flex radially inwardly and outwardly relative to the rest of the frame. Lateral arms can also couple upper ends of the commissure portions to adjacent apices to pull them in at larger diameters. The radial position of the commissure portions relative to the rest of the frame can affect the positioning of the leaflets and the effectiveness of the valve.

### CROSS-REFERENCE TO RELATED APPLICATIONS

This application is a continuation of International Patent Application No. PCT/US2022/037864, filed Jul. 21, 2022, which claims the benefit of U.S. Provisional Patent Application No. 63/226,328 filed Jul. 28, 2021. The prior applications are incorporated by reference herein in their entirety.

### FIELD

The present disclosure relates to prosthetic heart valves and frames therefor.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. Some prosthetic heart valves can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can resiliently self-expand to its functional size.

### SUMMARY

Described herein are prosthetic heart valves, delivery apparatus, and methods for implanting prosthetic heart valves. The disclosed prosthetic heart valves (and related delivery systems and methods) can include commissure portions that are radially offset and/or radially flexible relative to the rest of the frame. As such, the devices and methods disclosed herein can, among other things, overcome one or more of the deficiencies of typical prosthetic heart valves and their delivery apparatus.

In some examples, the commissure portions can extend radially outwardly from the rest of the frame such that they are positioned at a greater radius than the adjacent struts of the frame. The commissure portions of the frame can also be cantilevered from the frame, such that only one axial end of the commissure portions is attached to the rest of the frame, and the opposite axial end is free, allowing the commissure portions to flex radially inwardly and outwardly relative to the rest of the frame. The leaflets of a valve structure are secured to the commissure portions of the frame, and thus the radial position of the commissure portions relative to the rest of the frame can cause the leaflets to be more taught or more relaxed. When the commissure portions of the frame are radially offset outwardly from the rest of the frame, that can cause the articulation axes of the commissures to be positioned more radially even with the rest of the frame, enlarging the opening of the valve and reducing the pressure gradient across the valve during systole. In addition, the radial flexibility of the commissure portions allows the valve to function more efficiently across a wider range of implanted frame diameters, allowing the leaflets to have more slack when needed in situations where the frame expands to the larger end of the diameter range, and reducing excess slack in the leaflets when the frame is functioning at the small end of the diameter range.

Some exemplary frames comprise a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and an radially outer side face away from the central flow axis; wherein the plurality of interconnected struts are configured such that the frame is radially compressible to a compressed configuration and radially expandable to one or more expanded configurations; and the frame comprises commissure portions that are offset radially outwardly relative to the plurality of interconnected struts.

Some exemplary prosthetic heart valves comprise a frame and a leaflet valve structure positioned within the frame, wherein the frame comprises a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and an radially outer side face away from the central flow axis; and commissure portions that are offset radially outwardly relative to the plurality of interconnected struts; wherein the leaflet valve structure is positioned within the frame and secured to the commissure portions of the frame; and wherein the prosthetic heart valve is radially compressible to a compressed configuration and radially expandable to one or more expanded configurations.

Some exemplary delivery apparatuses comprises a handle portion, a shaft portion extending distally from the handle portion, and a prosthetic heart valve mounted at a distal portion of the shaft portion in a radially compressed configuration, wherein the prosthetic heart valve comprises an annular frame and a valve structure within the frame, and is radially expandable to one or more expanded configurations; wherein the frame comprises a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and an radially outer side face away from the central flow axis; and the frame comprises commissure portions that are offset radially outwardly relative to the plurality of interconnected struts, the commissure portions being secured to the valve structure; and wherein the delivery apparatus is configured to deliver the prosthetic heart valve to an implant location within a heart and to cause or allow the prosthetic heart valve to radially expand to the one or more expanded configurations at the implant location.

Some exemplary methods comprise: inserting a distal end of a delivery apparatus into the vasculature of a patient; advancing the prosthetic heart valve to a selected implantation site in the radially compressed state; actuating the handle to cause radial expansion of the prosthetic heart valve at the implantation side and disconnection of the prosthetic heart valve from the delivery device; and withdrawing the delivery device from the patient with prosthetic heart valve implanted at the implantation site.

Some exemplary frames comprise a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and an radially outer side face away from the central flow axis; wherein the plurality of interconnected struts are configured such that the frame is radially compressible to a compressed configuration and radially expandable to a first expanded configuration and a second expanded configuration, the second expanded configuration having a greater diameter than the first expanded configuration; wherein the plurality of interconnected struts form vertical columns of the frame that each have an inflow end apex and an outflow end apex, junctions connecting the vertical columns circumferentially to each other in a ring, commissure portions extending axially from the some of the junctions toward the outflow end of the frame, and lateral arms that extend circumferentially from outflow ends of the commissure portions to adjacent outflow end apices of the vertical columns.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of an exemplary frame for a prosthetic heart valve.

FIG. 2 is a top view of an exemplary prosthetic heart valve that includes the frame of FIG. 1, showing the valve structure in an open configuration.

FIG. 3 is a perspective view of an exemplary frame for a prosthetic heart valve, including radially offset commissure portions.

FIG. 4 is a side view of the frame of FIG. 3.

FIG. 5 is a top view of the frame of FIG. 3.

FIG. 6 is a perspective view of another exemplary frame for a prosthetic heart valve including radially offset commissure portions.

FIG. 7 is a side view of the frame of FIG. 6.

FIG. 8 is a top view of the frame of FIG. 6.

FIG. 9 is a perspective view of yet another exemplary frame for a prosthetic heart valve, including radially offset commissure portions.

FIG. 10 is a side view of the frame of FIG. 9.

FIG. 11 is a top view of the frame of FIG. 9.

FIG. 12 is a plan view of another exemplary frame for a prosthetic heart valve, where the tubular frame is cut and unrolled to a flattened position.

FIG. 13 is a perspective view of a frame according to the plan of FIG. 12.

FIGS. 14 and 15 are perspective views of portions of the frame of FIG. 13.

FIG. 16 is a top view of a prosthetic heart valve that includes the frame of FIG. 3 along with an inner leaflet valve structure coupled to the frame.

FIG. 17 is a perspective view of the prosthetic heart valve of FIG. 16.

FIG. 18 is a side view of an exemplary frame for a prosthetic heart valve that includes a screw actuation mechanism.

FIG. 19 is a side view of an exemplary prosthetic heart valve that includes a frame, a valve structure, and a skirt.

FIG. 20 is a schematic illustration of a device for delivering a prosthetic heart valve within a patient's heart.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Features, integers, characteristics, compounds, chemical moieties, or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any examples disclosed herein. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” The terms “a plurality of” and “plural” mean two or more of the specified element.

The term “coupled” generally means physically, mechanically, chemically, magnetically, and/or electrically coupled or linked and does not exclude the presence of intermediate elements between the coupled or associated items absent specific contrary language.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

In the context of the present application, the terms “lower” and “upper” are used interchangeably with the term's “inflow” and “outflow”, respectively. Thus, for example, the lower end of the valve is its inflow end and the upper end of the valve is its outflow end.

As used herein, with reference to the prosthetic heart valve and the delivery apparatus, “proximal” refers to a position, direction, or portion of a component that is closer to the user and/or a handle of the delivery apparatus that is outside the patient, while “distal” refers to a position, direction, or portion of a component that is further away from the user and/or the handle of the delivery apparatus and closer to the implantation site. The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the longitudinal axis of the prosthetic valve).

In view of the many possible embodiments to which the principles of the disclosed technology may be applied, it should be recognized that the illustrated examples are only preferred examples of the technology and should not be taken as limiting the scope of the invention(s). Rather, the scope of the invention(s) is at least as broad as the full scope of the appended claims.

### EXAMPLES OF THE DISCLOSED TECHNOLOGY

Described herein are examples of prosthetic heart valves, annular frames for prosthetic heart valves, leaflet assemblies for prosthetic heart valves, skirts and other additional components of prosthetic heart valves, delivery devices for implanting prosthetic heart valves, and related methods. The illustrated prosthetic valves are adapted to be implanted in the native aortic heart valve region, although in other examples it can be adapted to be implanted in the other native heart valve regions of the heart (for example, the pulmonary, mitral, and tricuspid valves). The prosthetic valves can also be adapted to be implanted in other tubular organs or passageways in the body. The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

Prosthetic heart valves can comprise an annular frame that includes a plurality of struts, and a valvular structure coupled within the frame. The valvular structure can comprise a plurality of leaflets, such as three leaflets, that open and close to regulate blood flow through the frame. In some examples, one or more skirts, sealing members, or other components can also be coupled to the frame and/or the leaflets to provide sealing or other functions. The valvular structure can comprise three leaflets, collectively forming a leaflet structure, which can be arranged to collapse in a tricuspid arrangement, although in other examples there can be greater or fewer number of leaflets (for example, one or more leaflets). In some examples, the leaflets can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

Prosthetic valves disclosed herein can be radially compressible and expandable between a radially compressed configuration and a radially expanded configuration. Thus, the prosthetic valves can be crimped onto a delivery apparatus in the radially compressed configuration during delivery, and then expanded to the radially expanded configuration once the prosthetic valve reaches the implantation site. In some examples, the prosthetic valve can be deployed from the delivery apparatus at the implantation site (for example, a native valve region of a heart) via inflating an inflatable balloon of the delivery apparatus. In other examples, the frame can comprise a shape memory material that allows the prosthetic valve to self-expand when released from confinement by the delivery device. In other examples, the prosthetic valve and/or the delivery device can include mechanical actuation mechanisms that expand the frame at the implantation site.

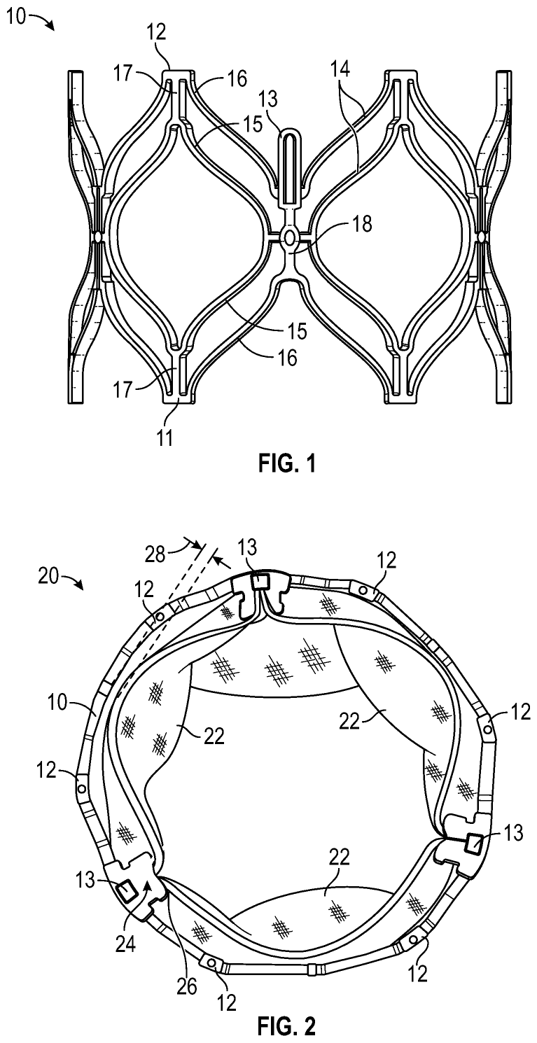

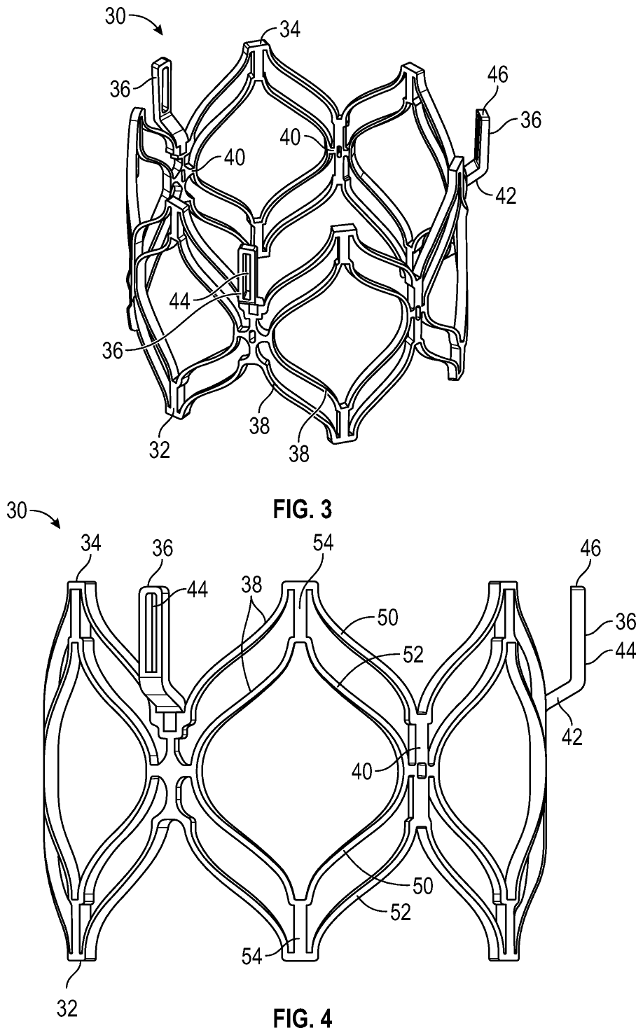

FIG. 1 shows one side of an exemplary annular frame 10 for a prosthetic heart valve. The frame 10 has an inflow end 11 and an outflow end 12. The frame 10 comprises three commissure portions 13 and a plurality of struts 14 that form six generally diamond-shaped vertical columns extending between the inflow end 11 and the outflow end 12, with two between each pair of commissure portions. Though only one is shown in FIG. 1, the frame 10 includes three commissure portions 13 equally spaced around the frame, the ends of which are shown in FIG. 2. Each of the six vertical columns is made up of several of the struts 14, including inner struts 15, outer struts 16, and vertical connector struts 17. The inner struts 15 of each vertical column form an inner cell that is generally diamond shaped. The outer struts 16 are spaced axially apart from the inner struts 15 and define apexes of the vertical columns at the inflow end 11 and outflow end 12 of the frame. The six outflow end apexes are show in FIG. 2 with the reference number 12. As shown in FIG. 1, the connector struts 17 extends axially from axial ends of the inner struts 15 to the apexes where they connect to the outer struts 16. The frame 10 also includes six cross-shaped junctions 18 positioned between the six vertical columns mid-way between the inflow end 11 and the outflow end 12. Each of the junctions 18 comprises lateral segments that connect circumferentially to the inner struts 15 of the two adjacent vertical columns, and vertical segments that connect axially to the outer struts 16 of the two adjacent vertical columns. Three of the six junctions 18 adjacent the commissure portions 13, and the other three are between the commissure portions. The commissure portions 13 extend axially toward the outflow end 12 from the junctions 18 and from between ends of adjacent outer struts 16. The commissure portions 13 are cantilevered such that free ends of the commissure portions can flex radially relative to the rest of the frame.

The valvular structure secured within the frame can comprise a plurality of leaflets, though other valve structures can be used as well. For example, FIG. 2 shows a top view of an exemplary prosthetic heart valve 20 that comprises the frame 10 of FIG. 1 along with a valvular structure that comprises three leaflets 22 that are coupled to the commissure portions 13 of the frame at commissures 24. The commissures are typically disposed within the outflow half of the frame, closer to the outflow end than the inflow end. The leaflets can also be coupled to the frame at a scallop-line that is closer the inflow end of the valve than the commissures (see the valve 400 of FIG. 19 for example).

The leaflets of a valve can cycle between closed and open states during the diastolic and systolic phases, and desirably minimize pressure gradients across the valve when open during the systolic phase, yet properly coapt with each other during diastole. The leaflets conventionally articulate about articulation axes of the commissure, which can be offset radially inwardly with respect to the annular frame. For example, in the valve 20, the leaflets 22 articulate about articulation axes that are located at the inner sides of the commissures 24, and the articulation axes are thus offset radially inward from the commissure portions 13 of the frame 10. This radial offsetting distance between the articulation axes and the frame can reduce the risk of the mid-portion of the leaflets (portions of the leaflets midway between two commissures) hitting against the inner surfaces of the frame during systole. For example, in the example of FIG. 2, the mid-portions of the leaflets 22 are still spaced slightly apart from the frame by a distance 28 when valve is fully open during systole.

However, some prosthetic valves that are designed to operate in a relatively wide range of frame diameters when implanted (for example, from 20 mm to 30 mm) This range of frame diameters can result in the leaflets being either overly tensioned or overly slacked when implanted. The leaflets can be overly tensioned when the frame is expanded to the higher end of the range of diameters (for example, 30 mm), and the leaflets can form excess slack if the frame is expanded to the smaller end of the range of diameters (for example, 20 mm) In the former case, the taught leaflets may not be able to close properly during diastole. In the latter case, the slack of leaflet material can result in excessive flow obstruction and a higher pressure gradient across the valve in the systolic phase. Thus, the relative radial position between the leaflets and the frame at different expansion diameters, for the same leaflet size, can be a limiting factor in determining the working range of a particular valve.

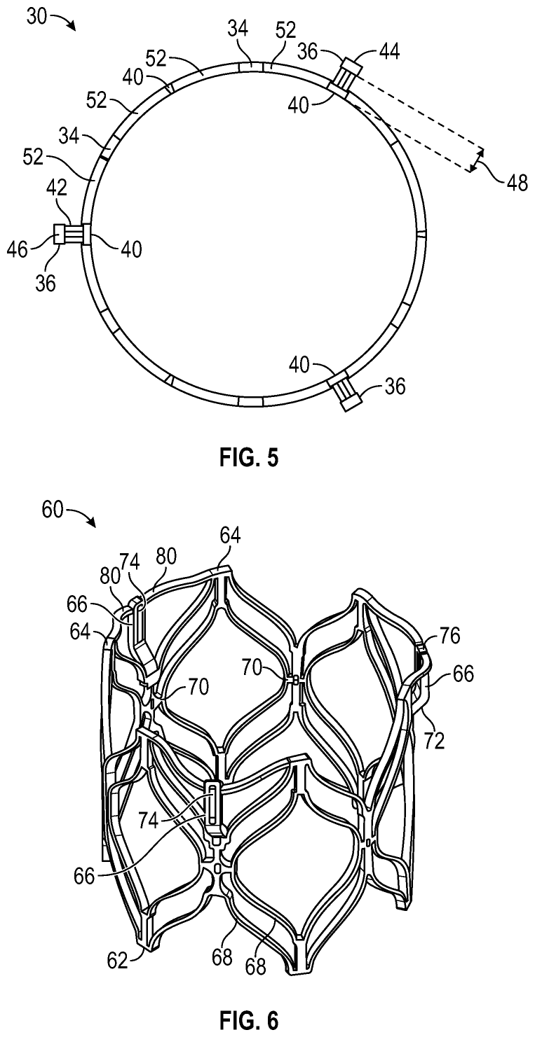

In some examples disclosed herein, a prosthetic heart valve comprises a frame with radially offset commissure portions. Such a frame can include commissure portions that are offset radially outwardly with respect to the strut portions of the frame that extend between the commissure portions. For example, FIGS. 3-5 illustrate a frame 30 that comprises radially offset commissure portions 36. The frame 30 is otherwise similar to the frame 10. The frame 30 has inflow end apices 32 and outflow end apices 34. A plurality of struts 38 form six generally diamond-shaped columns that extend vertically between a respective inflow end apex 32 and outflow end apex 34. Each vertical column comprises outer struts 50, inner struts 52, and vertical connector struts 54, like in the frame 10. The six vertical columns are joined at their lateral sides by six mid-frame junctions 40. The three commissure portions 36 extend in a cantilevered manner from to upper sides of three of the mid-frame junctions 40 between the vertical columns. Each of the commissure portions 36 includes an offsetting portion 42 extending radially outwardly from the respective mid-frame junction 40, and a vertical window portion 44 extending upwardly from the outer end of the offsetting portion to an upper end 46. The offsetting portion 42 results in the commissure portions 36 being radially offset a distance 48 (FIG. 5) from the rest of the frame. The distance 48 can be between 0 mm and 10 mm, between 0 mm and 5 mm, between 1 mm and 5 mm, between 2 mm and 4 mm, and/or about 3 mm. The window portions 44 can comprise two vertical struts with an opening between them, and are configured to be sutured to lateral tabs of the leaflets, forming the commissures of the valve.

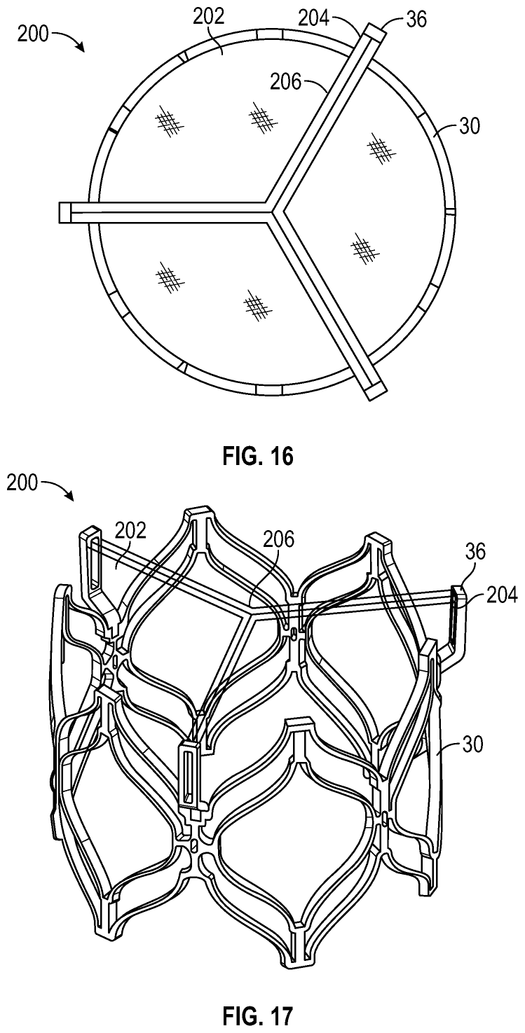

Because the window portions 44 are offset radially outwardly relative to the rest of the frame 30, the resulting valve commissures attached to the window portions 44 are also offset radially outwardly (relative to the non-offset configuration of FIG. 2). FIGS. 16 and 17 illustrate a prosthetic heart valve 200 that includes the frame 30 with offset commissure portions 36 and valve leaflets 202 attached to the commissure portions. Only the upper outflow edges 206 of the leaflets 202 are shown schematically in FIG. 17 for illustrative purposes. The leaflets 202 are illustrated in their closed position with the outflow edges 206 of the leaflets sealed together. In comparison to the valve 20 shown in FIG. 2, the offset commissure portions 36 cause the commissures to be radially outwardly offset such that the articulation axes of the leaflets 202 are moved radially outwardly (closer to the radial level of the rest of the frame) and the leaflets are pulled apart slightly, thereby allowing the leaflets to open wider (relative to a frame of the same diameter with non-offset commissure portions, like in FIG. 2) and reducing the amount slack in the leaflets, so as to improve pressure-gradients across the valve 200 during systole.

The commissures of the leaflets 202 can be connected to the commissure portions 16, 36 of a frame of a prosthetic valve (for example, frame 12, 30, 60, 100) using any known techniques method, or mechanisms, such as disclosed in U.S. Pat. No. 9,393,110, U.S. Publication No. 2018/0325665, or U.S. Application No. 63/003,085, filed Mar. 31, 2020, which are incorporated herein by reference. In particular examples, the leaflets 202 have a configuration and are connected to the frame at the commissures as disclosed in co-pending U.S. Application No. 63/224,534, filed Jul. 22, 2021 (Attorney Docket No. 11196US01), which is incorporated herein by reference.

In some examples, the commissure portions of a frame can be sufficiently flexible such that they are radially movable relative to the rest of the frame as the frame changes diameter. For example, the commissure portions of a frame can be movable between a radially outwardly offset configuration when the frame is at smaller expansion diameters (such as the positions shown in FIGS. 3-5 and 16-17) and an aligned configuration when the frame is at larger expansion diameters (where the commissure portions are generally in-line with the rest of the frame and not offset). This can allow the prosthetic valve to perform better over a larger range of implanted diameters.

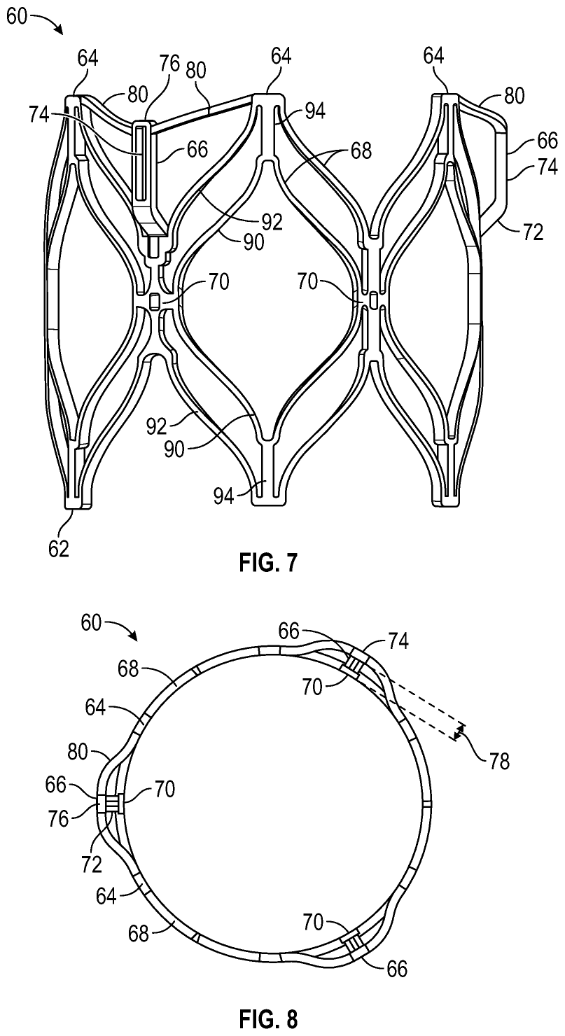

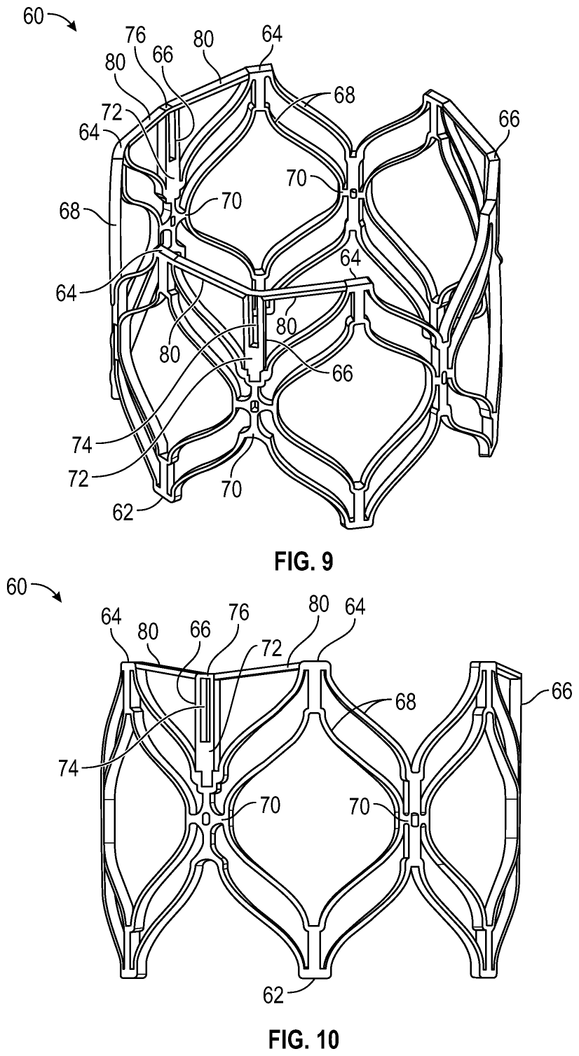

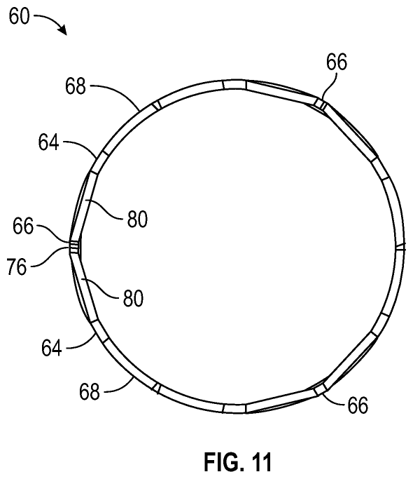

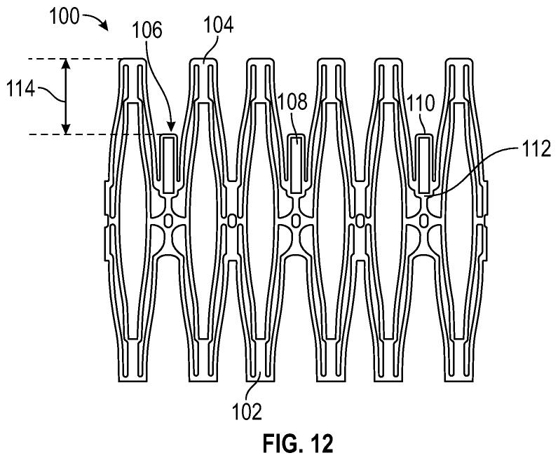

FIGS. 6-11 illustrates an exemplary frame 60 that has commissure portions 66 that are radially movable relative to the rest of the frame as the frame changes diameter. The frame 60 has six inflow end apices 62 and six outflow end apices 64. A plurality of struts 68 form six generally diamond-shaped columns that extend vertically between a respective inflow end apex 62 and outflow end apex 64. The six columns are joined at their sides by six mid-frame junctions 70. The vertical columns include inner struts 90, outer struts 92, and vertical connector struts 94, line in the frames 10 and 30. The three commissure portions 66 extend from upper sides of three of the mid-frame junctions 70 between the outer struts 92. Each of the commissure portions 66 includes an offsetting portion 72 extending radially outwardly from the respective mid-frame junction 70, a vertical window portion 74 extending upwardly from the outer end of the offsetting portion, and two lateral arms 80 that extend from an upper end 76 of the window portion to the two adjacent outflow end apices 64. The window portions 44 can comprise two vertical struts with an opening between them, and are configured to be sutured to lateral tabs of the leaflets, forming the commissures of the valve.

The radial offset distance 78 (FIG. 8) of the commissure portions 66 can vary depending on the expansion diameter of the frame 60. The distance 78 can vary between 0 mm and 10 mm, between 0 mm and 5 mm, between 0 mm and 4 mm, and/or between 0 mm and 3 mm A maximum value of the distance 78 (for example, when the frame is at a small expansion diameter) can be between 0 mm and 10 mm, between 1 mm and 5 mm, between 2 mm and 4 mm, and/or about 3 mm Both the offsetting portion 72 and the lateral arms 80 can flex relative to the rest of the frame to transition between a bent configuration, as shown in FIGS. 6-8 (for example, when the frame is at smaller expansion diameters), and a straightened configuration, as shown in FIGS. 9-11 (for example, when the frame is at larger expansion diameters). The offsetting portion 72 and the lateral arms 80 can comprise plastically deformable material (for example, MP35N) or shape-memory material (for example, NiTi), and can be shape set to assume an offset configuration in a free state at lower valve expansion diameters such as 26 mm (for example, FIGS. 6-8). In the offset configuration, the commissure window portions 74 are retained in a pre-shaped radially offset configuration, to achieve the same advantages described above with respect to the frame 30 of FIGS. 3-5.

As shown in FIGS. 9-11, when the frame 60 expands to larger diameters, for example to 29 mm, the outflow end apices 64 move apart from each other along the circumference of the frame, and pull the lateral arms 80 attached to the outflow end apices circumferentially, thereby transitioning the lateral arms to a straightened horizontal configuration, which in turn pulls the commissure window portions 74 radially inwardly and upwardly. The offsetting portions 72 are also passively pulled radially inwardly and upwardly, pivoting relative to the mid-frame junctions 70, to cause the whole commissure portions 66 to straighten and align with the circumferential level of the rest of the frame. Because the commissure portions 66 move radially inwardly relative to the rest of the frame as the frame expands to larger diameters, the leaflets are not pulled quite as far outwardly and can maintain sufficient working functionality at larger frame diameters that would otherwise be possible. Thus, the ability of the commissure portions to move radially relative to the rest of the frame as the frame changes diameter allows the prosthetic valve utilizing the frame to remain functional, and to function at a high level of performance, over a broader range of frame expansion diameter than would otherwise be afforded by a conventional valve frame where the commissure portions remains at equal radius with the rest of the frame.

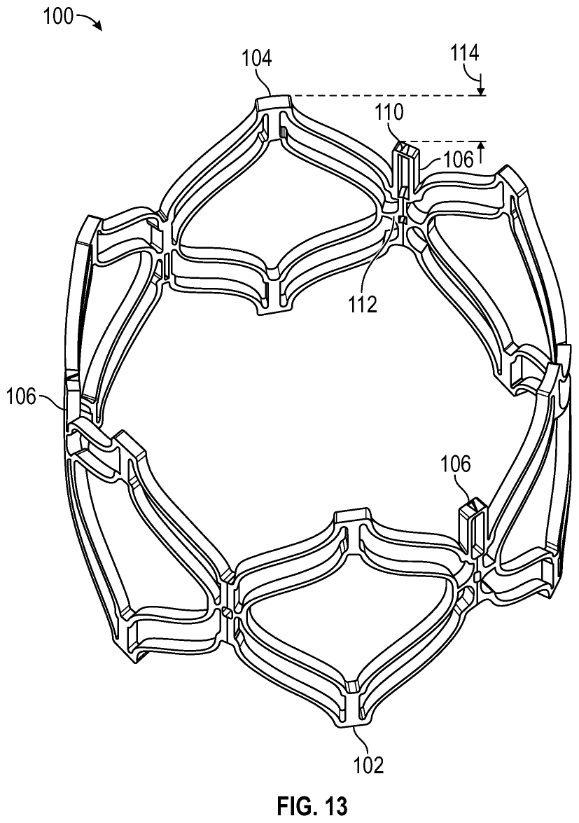

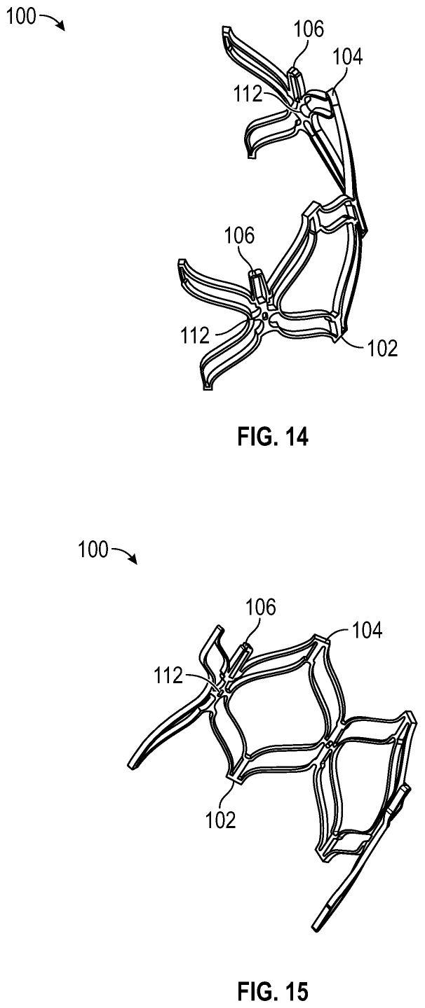

FIGS. 12-15 illustrate another exemplary frame 100 for a prosthetic heart valve. The frame 100 is shown a flattened and compressed layout in FIG. 12 to show the relative axial positions of the frame parts. FIGS. 14 and 15 show portions of the frame 100. The frame 100 comprises six generally diamond shaped columns extending between inflow end apices 102 and outflow end apices 104, and connected laterally by mid-frame junctions 112. The frame 100 includes three cantilevered commissure portions 106, which include an opening 108 between vertical struts for connection to the leaflets. As shown in FIG. 12, the upper ends 110 of the commissure portions 106 are spaced axially a substantial distance 114 below the outflow end apices 104. The distance 114 can be between 0 mm and 20 mm, between 1 mm and 20 mm, between 2 mm and 15 mm, between 3 mm and 12 mm, and/or between 5 mm and 10 mm Since the whole region of commissure attachment is spaced further away from the outflow end of the frame, the leaflets can be much shorter in axial height, which in turn can reduce pressure gradients across the valve. Moreover, since the commissure portions are attached to the junctions 112 at their lower ends while all other edges thereof are free, the commissure portions can be somewhat bendable radially, enabling it to bend radially inward during diastole. A further advantage of axially shorter leaflets is that they can allow for greater access to the coronary access when the valve is implanted at the aortic valve region of the heart, for example in ViV procedures, as the shorter leaflets terminating at edges that are distal to the frame's outflow end, present a lower risk of curtaining the coronary ostia. Small leaflets can also help reduce the overall crimp profile of the valve during delivery.

In any the disclosed frames where the commissure portions are cantilevered from the mid-frame junctions with their upper ends free (for example, frame 10 in FIGS. 1-2, frame 30 in FIGS. 3-5 & 16-17, and frame 100 in FIGS. 12-15), the commissure portions can be bendable radially inwardly relative to the rest of the frame. This can allow the commissure portions to bend radially inwardly during diastole and help the leaflets close properly, and can also allow the commissure portions to move back radially outwardly during systole to help the valve open wide and reduce pressure gradient across the valve. The inward flexibility of the commissure portions can also allow the leaflets to be shorter and narrower. Thus, leaflets can be designed for a smaller valve diameter, exploiting the flexible commissure portion's contribution to their closure during diastole.

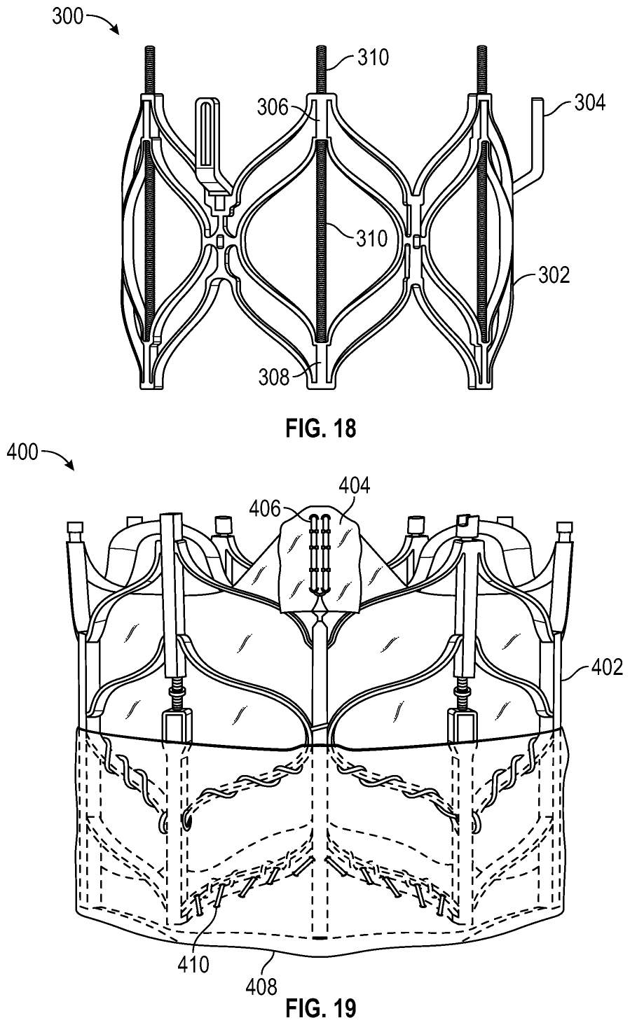

FIG. 18 shows an exemplary frame 300 that is a variation of the frame 30, wherein radial expansion of the frame is accomplished via one or more actuators comprising one or more screws or threaded rods 310. The frame 300 comprises a strut structure 302 similar to the frame 30, with radially offset commissure portions 304, inflow end apices 308, outflow end apices 306, and a plurality of screws 310 that extend axially through the outflow end apices 306, across the diamond shaped cells, and into the inflow end apices 308. The screws 310 are threadedly engaged with the outflow end apices 306, such that when the screws are rotated the outflow end apices 306 move axially along the screws toward the inflow end apices 308 to axially shorten the frame and thereby radially expand the frame. The screws can also be rotated the opposite direction to radially collapse the frame. In other examples, the frame 300 can include other types of actuators to produce radial expansion and compression of the frame, such as reciprocating or push-pull actuators, which can include locking mechanisms that can retain the frame at a desired expanded diameter after it is radial expanded inside a patient. More descriptions of mechanical actuators for controlling expansion of prosthetic valves, and related delivery devices, can be found in U.S. Publication Nos. 2018/0153689, 2018/0311039, 2019/0060057, International Application Nos. PCT/US2020/0057691, filed Oct. 28, 2020, PCT/US2020/063104, filed Dec. 3, 2020, U.S. Provisional Patent Application No. 63/085,947 filed Sep. 30, 2020, and U.S. Provisional Application No. 62/990,299, filed Mar. 16, 2020, all of which are incorporated by reference herein.

Any of the frames disclosed herein can be adapted for various manners of radial expansion, such as being adapted to include screws similar to the frame 300 for screw-actuated expansion other types of mechanical actuators (for example, push-pull actuators), being adapted for balloon actuated expansion (for example, wherein the frame can be made of a plastically deformable material), or being adapted for self-expansion (for example, wherein the frame can be made of superelastic or shape memory material). The frames disclosed herein can be made of any of various suitable plastically-expandable (plastically-deformable) materials (for example, stainless steel, etc.) or self-expanding materials (for example, nickel titanium alloy (NiTi), such as nitinol). When constructed of a plastically-expandable material, the frames (and thus the prosthetic valves) can be crimped to a radially collapsed configuration on a delivery apparatus (for example, catheter) and then expanded inside a patient by an inflatable balloon or equivalent expansion mechanism. When constructed of a self-expandable material, the frames (and thus the prosthetic valves) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Suitable plastically-expandable materials that can be used to form the frames include, without limitation, stainless steel, a biocompatible, high-strength alloys (for example, a cobalt-chromium or a nickel-cobalt-chromium alloys), polymers, or combinations thereof. In particular examples, frames can be made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

FIG. 19 illustrates an exemplary prosthetic heart valve 400 that comprises a frame 402 and a leaflet structure 404 coupled to commissure portions of the frame via sutures 406. The valve 400 also comprises an outer skirt, or paravalvular sealing member, 408 that is secured to the frame 402 via sutures 410. Such an outer skirt can comprise a fabric material, tissue material, or similar flexible and flow-proof material, and can help seal the region between the outside of the frame and the native tissue against which the valve is implanted to prevent paravalvular blood flow. The outer skirt can also promote tissue ingrowth and help secure the valve to the tissue. Any of the herein disclosed frames can be used with such an outer skirt. Further details of the prosthetic valve 400 are disclosed in U.S. Application No. 63/085,947, filed Sep. 30, 2020, which is incorporated herein by reference.

For any prosthetic valve utilizing the herein disclosed frames, each leaflet of the internal valve structure can be coupled to the frame at the commissure portions of the frame and also coupled to the frame along its inflow edge (the lower edge in the figures; also referred to as “cusp edges”). In some examples, a reinforcing element or connecting skirt, such as a fabric strip, can be connected directly to the cusp edges of the leaflets and to the struts of the frame to couple the cusp edges of the leaflets to the frame (for example, as shown in FIG. 19).

Further, any of the frames disclosed herein can, in some examples, have a cylindrical or substantially cylindrical shape having a constant diameter from the inflow end to the outflow end of the frame, or the frame can, in other examples, vary in diameter along the height of the frame, as disclosed in U.S. Patent Publication No. 2012/0239142, which is incorporated herein by reference.

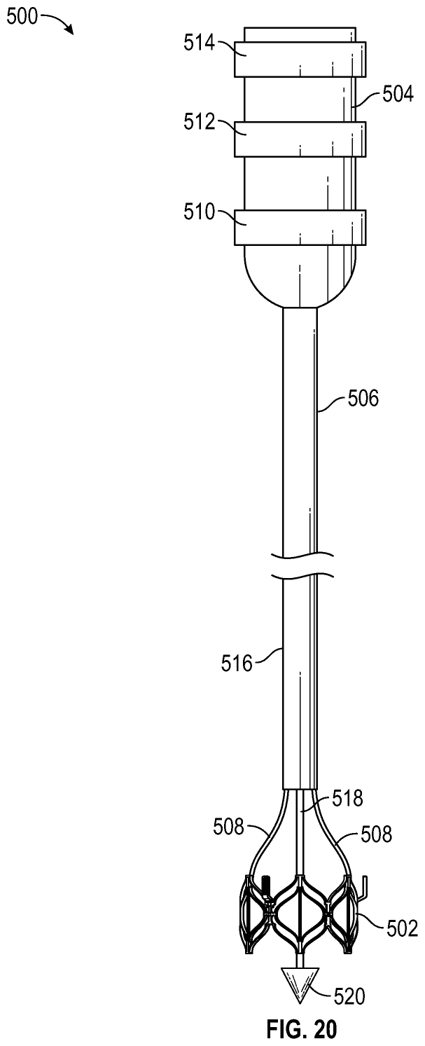

FIG. 20 illustrates an exemplary delivery apparatus 500 that can be used to delivery any of the prosthetic valves disclosed herein into a heart. An example prosthetic valve 502 is represented by an example frame (similar to frame 300) only in FIG. 20 for illustrative purposes, though a complete prosthetic valve incorporating such a frame (along with a leaflet structure and other components) would be positioned where just the frame is illustrated. The prosthetic valve 502 can be releasably coupled to the delivery apparatus 500. The delivery apparatus 500 and other delivery apparatuses disclosed herein can be used to implant analogous prosthetic devices other than prosthetic heart valves, such as stents or grafts and other types of valves.

The delivery apparatus 500 in the illustrated example generally includes a handle 504, a first elongated shaft 506 (which comprises an outer shaft in the illustrated example) extending distally from the handle 504, at least one actuator assembly 508 extending distally through the outer shaft 506. The at least one actuator assembly 508 can be configured to radially expand and/or radially collapse the prosthetic valve 502 when actuated (for example, via screw actuators or otherwise).

Though the illustrated example shows two actuator assemblies 508 for purposes of illustration, it should be understood that one actuator 508 can be provided for each actuator on the prosthetic valve. For example, three actuator assemblies 508 can be provided for a prosthetic valve having three actuators. In other examples, a greater or fewer number of actuator assemblies can be present.

In other examples, the delivery apparatus can comprise an inflatable balloon positioned within the prosthetic valve and means for inflating the balloon to expand the valve, instead of or in addition to the actuator assemblies 508.

In some examples, a distal end portion 516 of the shaft 506 can be sized to house the prosthetic valve 502 in its radially compressed, delivery state during delivery of the prosthetic valve through the patient's vasculature. In this manner, the distal end portion 516 functions as a delivery sheath or capsule for the prosthetic valve during delivery,

In examples that include actuator assemblies 508, the actuator assemblies can be releasably coupled to the prosthetic valve 502. For example, in the illustrated example, each actuator assembly 508 can be coupled to a respective actuator of the prosthetic valve 502. For example, each actuator assembly 508 can comprise a support tube and an actuator member (for example, a rotatable actuator member or driver) extending through the support tube. The support tube has a distal end portion that can abut an adjacent section of the frame 200. The rotatable driver has a distal end portion that can engage a respective screw 310. The rotatable driver and the support can cooperate to form a releasable connection with the screw 310.

When actuated, the actuator assembly can transmit rotational or pushing and/or pulling forces to portions of the prosthetic valve to radially expand and collapse the prosthetic valve as previously described. The actuator assemblies 508 can be at least partially disposed radially within, and extend axially through, one or more lumens of the outer shaft 506. For example, the actuator assemblies 508 can extend through a central lumen of the shaft 506 or through separate respective lumens formed in the shaft 506.

The handle 504 of the delivery apparatus 500 can include one or more control mechanisms (for example, knobs, levers, dials, buttons, or other actuating mechanisms) for controlling different components of the delivery apparatus 500 in order to expand and/or deploy the prosthetic valve 502. For example, in the illustrated example the handle 504 comprises first, second, and third knobs 510, 512, and 514.

In some examples, the first knob 510 can be a rotatable knob configured to produce axial movement of the outer shaft 506 relative to the prosthetic valve 502 in the distal and/or proximal directions in order to deploy the prosthetic valve from the delivery sheath 516 once the prosthetic valve has been advanced to a location at or adjacent the desired implantation location with the patient's body. For example, rotation of the first knob 510 in a first direction (for example, clockwise) can retract the sheath 516 proximally relative to the prosthetic valve 502 and rotation of the first knob 510 in a second direction (for example, counter-clockwise) can advance the sheath 516 distally. In other examples, the first knob 510 can be actuated by sliding or moving the knob 510 axially, such as pulling and/or pushing the knob. In other examples, actuation of the first knob 510 (rotation or sliding movement of the knob 510) can produce axial movement of the actuator assemblies 508 (and therefore the prosthetic valve 502) relative to the delivery sheath 516 to advance the prosthetic valve distally from the sheath 516.

The second knob 512 can be a rotatable knob configured to produce radial expansion and/or contraction of the prosthetic valve 502. For example, the proximal end portions of the drivers can be operatively coupled to the second knob 512 such that rotation of the second knob can rotate the drivers relative to the support tubes and produce corresponding rotation of the threaded rods 310 of the prosthetic valve. Rotation of the second knob 512 in a first direction (for example, clockwise) can radially expand the prosthetic valve 502 and rotation of the second knob 512 in a second direction (for example, counter-clockwise) can radially collapse the prosthetic valve 502. In other examples, for example, if the actuators of the prosthetic valve are reciprocating type actuators, the second knob 512 can be actuated by sliding or moving the knob 512 axially, such as pulling and/or pushing the knob.

The third knob 514 can be a rotatable knob configured to release the prosthetic heart valve 502 from the delivery device 500. For example, the proximal end portions of the support tubes can be operatively coupled to the third knob 514 such that rotation of the third knob 514 produces axial movement of the support tubes relative to the drivers. Rotation of the third knob in a first direction (for example, clockwise) can move the support tubes in a proximal direction relative to the drivers and away from the prosthetic valve. Once the distal end portions of the support tubes are retracted proximally beyond the distal end portions of the drivers that engage the threaded rods 310, the distal end portions of the drivers can be configured to automatically disengage from the threaded rods 310 In other examples, the third knob 514 can be actuated by sliding or moving the third knob 514 axially, such as pulling and/or pushing the knob. Further details regarding the delivery device 500 are disclosed in U.S. Application No. 63/085,947, filed Sep. 30, 2020, which is incorporated herein by reference. Other delivery devices can be used to deliver and implant the prosthetic valves disclosed herein, such as those disclosed in PCT Application No. PCT/US2020/063104, filed Dec. 3, 2020, and U.S. Application No. 62/990,299, filed Mar. 16, 2020, which are incorporated herein by reference.

A method of delivering prosthetic valve 502 generally includes placing prosthetic valve 502 in a radially compressed state, for example, by operating the actuators of the prosthetic valve to place the frame in a radially compressed configuration. This can be accomplished by releasably coupling the prosthetic valve to the actuator assemblies 508 of the delivery device and actuating the second knob 512. The radially compressed prosthetic valve optionally can be placed within a sheath 516 of the delivery device. The delivery device and prosthetic device can be advanced over a guidewire through the vasculature of a patient to a selected implantation site (for example, the native aortic annulus). For example, when implanting the prosthetic valve within the native aortic valve, the delivery device and prosthetic valve can be inserted into and through a femoral artery, and through the aorta to the native aortic valve. At the implantation site, if initially contained within a sheath 516, the prosthetic valve 502 can be deployed from the sheath 516 by actuating the first knob 510. The prosthetic valve 502 can then be radially expanded to a desired size by actuating the second knob 512. Once the prosthetic device is at the desired diameter, the actuator assemblies of the delivery device are uncoupled from the prosthetic valve by actuating the third knob 514, allowing removal of the delivery device from the patient's body.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam.

Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### ADDITIONAL EXAMPLES OF THE DISCLOSED TECHNOLOGY

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A frame for a prosthetic heart valve comprising:

a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis;

wherein the plurality of interconnected struts are configured such that the frame is radially compressible to a compressed configuration and radially expandable to one or more expanded configurations; and

commissure portions that are offset radially outwardly relative to the plurality of interconnected struts.

Example 2. The frame of any example herein, particularly example 1, wherein the commissure portions are coupled to the plurality of interconnected struts at an inflow end of the commissure portions.

Example 3. The frame of any example herein, particularly any one of examples 1-2, wherein the commissure portions comprise a leaflet attachment portion comprising an opening defined between two axial struts of the commissure portions.

Example 4. The frame of any example herein, particularly any one of examples 1-3, wherein the commissure portions comprise an axially extending leaflet attachment portion and an offsetting portion that extends radially outwardly from the plurality of interconnected struts to the leaflet attachment portion.

Example 5. The frame of any example herein, particularly example 4, wherein the offsetting portion extends both radially outwardly and axially toward the outflow end from the plurality of interconnected struts to the leaflet attachment portion.

Example 6. The frame of any example herein, particularly any one of examples 1-5, wherein the plurality of interconnected struts comprises mid-frame junctions and the commissure portions extend from the mid-frame junctions.

Example 7. The frame of any example herein, particularly any one of examples 1-6, wherein the frame comprises an inflow half adjacent the inflow end and an outflow half adjacent the outflow end, and the commissure portions are positioned in the outflow half.

Example 8. The frame of any example herein, particularly any one of examples 1-7, wherein the commissure portions define a maximum radial extent of the frame.

Example 9. The frame of any example herein, particularly any one of examples 1-8, wherein the plurality of interconnected struts form six diamond-shaped cells that extend between the inflow end of the frame and the outflow end of the frame, wherein the six diamond-shaped cells are connected laterally by six mid-frame junctions positioned between the cells, and the commissure portions extend radially outwardly from three of the six mid-frame junctions.

Example 10. The frame of any example herein, particularly any one of examples 1-9, wherein the commissure portions are coupled to the plurality of interconnected struts at one axial end of the commissure portions and free from the plurality of interconnected struts at an opposite, free axial end of the commissure portions and at lateral sides of the commissure portions, such that the commissure portions are cantilevered.

Example 11. The frame of any example herein, particularly example 10, wherein the commissure portions terminate at the free axial ends, and the free axial ends are short of the outflow end of the frame, such that the free axial ends of the commissure portions are positioned axially between the inflow end of the frame and the outflow end of the frame.

Example 12. The frame of any example herein, particularly any one of examples 1-9, further comprising lateral arms that extend circumferentially from axial ends of the commissure portions to adjacent outflow end apices of the frame.

Example 13. The frame of any example herein, particularly example 12, wherein the frame comprises two of the lateral arms for each of the commissure portions.

Example 14. The frame of any example herein, particularly any one of examples 12-13, wherein when the frame is in a partially radially expanded state, the lateral arms extend both circumferentially and radially inwardly from axial ends of the commissure portions to adjacent outflow end apices of the frame.

Example 15. The frame of any example herein, particularly any one of examples 12-14, wherein the lateral arms pull the commissure portions radially inwardly, relative to the outflow end apices, as the outflow end apices move apart from each other when the frame radially expands.

Example 16. The frame of any example herein, particularly example 15, wherein when the frame is radially expanded to maximum extent, the commissure portions are radially even with the outflow end apices and the lateral arms extend horizontally between the axial ends of the commissure portions and the outflow end apices.

Example 17. The frame of any example herein, particularly any one of examples 1-16, wherein when the frame is in the expanded configuration, the commissure portions are configured to flex radially inwardly relative to the plurality of interconnected struts.

Example 18. The frame of any example herein, particularly any one of examples 1-17, wherein the commissure portions are offset radially outwardly between 2 mm and 4 mm relative to the plurality of interconnected struts.

Example 19. The frame of any example herein, particularly any one of examples 1-18, wherein the frame comprises shape memory material and is self-expandable.

Example 20. The frame of any example herein, particularly any one of examples 1-18, wherein the frame further comprises axially oriented screws coupled to the plurality of interconnected struts, and rotation of the screws causes the frame to axially shorten and radially expand.

Example 21. A prosthetic heart valve comprising:

a frame comprising:
a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis; and
commissure portions that are offset radially outwardly relative to the plurality of interconnected struts; and

a leaflet valve structure positioned within the frame and secured to the commissure portions of the frame;
wherein the prosthetic heart valve is radially compressible to a compressed configuration and radially expandable to one or more expanded configurations.

Example 22. The prosthetic heart valve of any example herein, particularly example 21, further comprising an outer skirt secured to an outer side of the frame adjacent the inflow end.

Example 23. The prosthetic heart valve of any example herein, particularly any one of examples 21-22, wherein commissures of the leaflet valve structure are positioned radially outwardly relative to the a plurality of interconnected struts.

Example 24. The prosthetic heart valve of any example herein, particularly any one of examples 21-23, wherein the leaflet valve structure articulates about articulation axes that located at a radial dimension that is equal to or greater than a radial dimension of the plurality of interconnected struts.

Example 25. The prosthetic heart valve of any example herein, particularly any one of examples 21-24, wherein scalloped inflow edges of the leaflet valve structure are secured to the plurality of interconnected struts.

Example 26. The prosthetic heart valve of any example herein, particularly example 25, wherein the commissure portions are coupled to the plurality of interconnected struts at an inflow end of the commissure portions.

Example 27. The prosthetic heart valve of any example herein, particularly any one of examples 21-26, wherein the commissure portions comprise a leaflet attachment portion comprising an opening defined between two axial struts of the commissure portions, and the leaflet valve structure extends through the opening and is secured to the axial struts.

Example 28. The prosthetic heart valve of any example herein, particularly any one of examples 21-27, wherein the commissure portions comprise an axially extending leaflet attachment portion and an offsetting portion that extends radially outwardly from the plurality of interconnected struts to the leaflet attachment portion.

Example 29. The prosthetic heart valve of any example herein, particularly example 28, wherein the offsetting portion extends both radially outwardly and axially toward the outflow end from the plurality of interconnected struts to the leaflet attachment portion.

Example 30. The prosthetic heart valve of any example herein, particularly any one of examples 21-29, wherein the plurality of interconnected struts comprises mid-frame junctions and the commissure portions extend from the mid-frame junctions.

Example 31. The prosthetic heart valve of any example herein, particularly any one of examples 21-30, wherein the frame comprises an inflow half adjacent the inflow end and an outflow half adjacent the outflow end, and the commissure portions are positioned in the outflow half.

Example 32. The prosthetic heart valve of any example herein, particularly any one of examples 21-31, wherein the commissure portions define a maximum radial extent of the frame.

Example 33. The prosthetic heart valve of any example herein, particularly any one of examples 21-32, wherein the plurality of interconnected struts form six diamond-shaped cells that extend between the inflow end of the frame and the outflow end of the frame, wherein the six diamond-shaped cells are connected laterally by six mid-frame junctions positioned between the cells, and the commissure portions extend radially outwardly from three of the six mid-frame junctions.

Example 34. The prosthetic heart valve of any example herein, particularly any one of examples 21-33, wherein the commissure portions are coupled to the plurality of interconnected struts at one axial end of the commissure portions and free from the plurality of interconnected struts at an opposite, free axial end of the commissure portions and at lateral sides of the commissure portions, such that the commissure portions are cantilevered.

Example 35. The prosthetic heart valve of any example herein, particularly example 34, wherein the commissure portions terminate at the free axial ends, and the free axial ends are short of the outflow end of the frame, such that the free axial ends of the commissure portions are positioned axially between the inflow end of the frame and the outflow end of the frame.

Example 36. The prosthetic heart valve of any example herein, particularly any one of examples 21-33, further comprising lateral arms that extend circumferentially from axial ends of the commissure portions to adjacent outflow end apices of the frame.

Example 37. The prosthetic heart valve of any example herein, particularly example 36, wherein the frame comprises two of the lateral arms extending from each of the commissure portions.

Example 38. The prosthetic heart valve of any example herein, particularly any one of examples 36-37, wherein when the frame is in a partially radially expanded state, the lateral arms extend both circumferentially and radially inwardly from axial ends of the commissure portions to adjacent outflow end apices of the frame.

Example 39. The prosthetic heart valve of any example herein, particularly any one of examples 36-38, wherein the lateral arms pull the commissure portions radially inwardly, relative to the outflow end apices, as the outflow end apices move apart from each other when the prosthetic heart valve radially expands.

Example 40. The prosthetic heart valve of any example herein, particularly example 39, wherein when the prosthetic heart valve is radially expanded to maximum extent, the commissure portions of the frame are radially even with the outflow end apices of the frame, and the lateral arms extend horizontally between the axial ends of the commissure portions and the outflow end apices.

Example 41. The prosthetic heart valve of any example herein, particularly any one of examples 21-40, wherein when the prosthetic heart valve is in an expanded configuration, the commissure portions of the frame are configured to flex radially inwardly relative to the plurality of interconnected struts when the leaflet valve structure closes during diastole to help the leaflet valve structure fully coapt and prevent retrograde flow.

Example 42. The prosthetic heart valve of any example herein, particularly any one of examples 21-41, wherein the commissure portions of the frame are offset radially outwardly between 2 mm and 4 mm relative to the plurality of interconnected struts.

Example 43. The prosthetic heart valve of any example herein, particularly any one of examples 21-42, wherein the frame comprises shape memory material and the prosthetic heart valve is self-expandable.

Example 44. The prosthetic heart valve of any example herein, particularly any one of examples 21-42, wherein the frame further comprises axially oriented screws coupled to the plurality of interconnected struts, and rotation of the screws causes the frame to axially shorten and causes the prosthetic heart valve to radially expand.

Example 45. A delivery apparatus comprising:

a handle portion;

a shaft portion extending distally from the handle portion; and

a prosthetic heart valve mounted at a distal portion of the shaft portion in a radially compressed configuration;

wherein the prosthetic heart valve comprises an annular frame and a valve structure within the frame, and is radially expandable to one or more expanded configurations;

wherein the frame comprises:
a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis; and
commissure portions that are offset radially outwardly relative to the plurality of interconnected struts, the commissure portions being secured to the valve structure; and

wherein the delivery apparatus is configured to deliver the prosthetic heart valve to an implant location within a heart and to cause or allow the prosthetic heart valve to radially expand to the one or more expanded configurations at the implant location.

Example 46. The delivery apparatus of any example herein, particularly example 45, wherein the shaft portion comprises a sheath that holds the prosthetic heart valve in the radially compressed configuration and the handle comprises an actuator that causes the sheath to move axially to uncover the prosthetic heart valve and allow the prosthetic heart valve to self-expand.

Example 47. The delivery apparatus of any example herein, particularly example 45, wherein the shaft portion comprises an inflatable balloon positioned within the radially compressed prosthetic heart valve and the delivery apparatus is configured to inflate the balloon to radially expand the prosthetic heart valve.

Example 48. The delivery apparatus of any example herein, particularly example 45, wherein the shaft portion comprises at least one actuator coupled to the frame of the prosthetic heart valve and the handle comprises a controller that causes the at least one actuator to turn at least one screw of the frame of the prosthetic heart valve to cause the prosthetic heart valve to radially expand.

Example 49. The delivery apparatus of any example herein, particularly any one of examples 45-48, wherein the prosthetic heart valve further comprises an outer skirt secured to an outer side of the frame adjacent the inflow end.

Example 50. The delivery apparatus of any example herein, particularly any one of examples 45-49, wherein commissures of the valve structure are positioned radially outwardly relative to the plurality of interconnected struts of the frame.

Example 51. The delivery apparatus of any example herein, particularly any one of examples 45-50, wherein the valve structure articulates about articulation axes that located at a radial dimension that is equal to or greater than a radial dimension of the plurality of interconnected struts.

Example 52. The delivery apparatus of any one of any example herein, particularly any one of examples 45-51, wherein scalloped inflow edges of the valve structure are secured to the plurality of interconnected struts.

Example 53. The delivery apparatus of any example herein, particularly any one of examples 45-52, wherein the commissure portions of the frame are coupled to the plurality of interconnected struts at an inflow end of the commissure portions.

Example 54. The delivery apparatus of any example herein, particularly any one of examples 45-53, wherein the commissure portions comprise a leaflet attachment portion comprising an opening defined between two axial struts of the commissure portions, and the leaflet valve structure extends through the opening and is secured to the axial struts.

Example 55. The delivery apparatus of any example herein, particularly any one of examples 45-54, wherein the commissure portions of the frame comprise an axially extending leaflet attachment portion and an offsetting portion that extends radially outwardly from the plurality of interconnected struts to the leaflet attachment portion.

Example 56. The delivery apparatus of any example herein, particularly example 55, wherein the offsetting portion extends both radially outwardly and axially toward the outflow end from the plurality of interconnected struts to the leaflet attachment portion.

Example 57. The delivery apparatus of any example herein, particularly any one of examples 45-56, wherein the plurality of interconnected struts comprises mid-frame junctions and the commissure portions extend from the mid-frame junctions.

Example 58. The delivery apparatus of any example herein, particularly any one of examples 45-57, wherein the frame comprises an inflow half adjacent the inflow end and an outflow half adjacent the outflow end, and the commissure portions are positioned in the outflow half.

Example 59. The delivery apparatus of any example herein, particularly any one of examples 45-58, wherein the commissure portions define a maximum radial extent of the frame.

Example 60. The delivery apparatus of any example herein, particularly any one of examples 45-59, wherein the plurality of interconnected struts form six diamond-shaped cells that extend between the inflow end of the frame and the outflow end of the frame, wherein the six diamond-shaped cells are connected laterally by six mid-frame junctions positioned between the cells, and the commissure portions extend radially outwardly from three of the six mid-frame junctions.

Example 61. The delivery apparatus of any example herein, particularly any one of examples 45-60, wherein the commissure portions are coupled to the plurality of interconnected struts at one axial end of the commissure portions and free from the plurality of interconnected struts at an opposite, free axial end of the commissure portions and at lateral sides of the commissure portions, such that the commissure portions are cantilevered.

Example 62. The delivery apparatus of any example herein, particularly example 61, wherein the commissure portions terminate at the free axial ends, and the free axial ends are short of the outflow end of the frame, such that the free axial ends of the commissure portions are positioned axially between the inflow end of the frame and the outflow end of the frame.

Example 63. The delivery apparatus of any example herein, particularly any one of examples 45-60, further comprising lateral arms that extend circumferentially from axial ends of the commissure portions to adjacent outflow end apices of the frame.

Example 64. The delivery apparatus of any example herein, particularly example 63, wherein the frame comprises two of the lateral arms extending from each of the commissure portions.

Example 65. The delivery apparatus of any example herein, particularly any one of examples 63-64, wherein when the frame is in a partially radially expanded state, the lateral arms extend both circumferentially and radially inwardly from axial ends of the commissure portions to adjacent outflow end apices of the frame.

Example 66. The delivery apparatus of any example herein, particularly any one of examples 63-65, wherein the lateral arms pull the commissure portions radially inwardly, relative to the outflow end apices, as the outflow end apices move apart from each other when the prosthetic heart valve radially expands.

Example 67. The delivery apparatus of any example herein, particularly example 66, wherein when the prosthetic heart valve is radially expanded to maximum extent, the commissure portions of the frame are radially even with the outflow end apices of the frame, and the lateral arms extend horizontally between the axial ends of the commissure portions and the outflow end apices.

Example 68. The delivery apparatus of any example herein, particularly any one of examples 45-67, wherein the commissure portions of the frame are configured to flex radially inwardly relative to the plurality of interconnected struts when the leaflet valve structure closes during diastole to help the leaflet valve structure fully coapt and prevent retrograde flow after implantation.

Example 69. The delivery apparatus of any example herein, particularly any one of examples 45-68, wherein the commissure portions of the frame are offset radially outwardly between 2 mm and 4 mm relative to the plurality of interconnected struts.

Example 70. The delivery apparatus of any example herein, particularly any one of examples 45-46 or 49-67, wherein the frame comprises shape memory material and the prosthetic heart valve is self-expandable.

Example 71. The delivery apparatus of any example herein, particularly any one of examples 45 or 48-67, wherein the frame further comprises axially oriented screws coupled to the plurality of interconnected struts, and rotation of the screws by the delivery apparatus causes the frame to axially shorten and causes the prosthetic heart valve to radially expand.

Example 72. A method comprising:

inserting a distal end of the delivery apparatus of any one of examples 45-71 into the vasculature of a patient;

advancing the prosthetic heart valve to a selected implantation site in the radially compressed state;

actuating the handle to cause radial expansion of the prosthetic heart valve at the implantation side and disconnection of the prosthetic heart valve from the delivery device; and

withdrawing the delivery device from the patient with prosthetic heart valve implanted at the implantation site.

Example 73. The method of any example herein, particularly example 72, wherein actuating the handle causes rotation of screws that axially shorten the frame and expand the prosthetic heart valve.

Example 74. The method of any example herein, particularly example 72, wherein actuating the handle causes axial motion of a sheath that uncovers the prosthetic heart valve and allows the prosthetic heart valve to self-expand.

Example 75. The method of any example herein, particularly example 72, wherein actuating the handle causes inflation of a balloon that radially expands the prosthetic heart valve.

Example 76. A frame for a prosthetic heart valve, the frame comprising:

a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis;

wherein the plurality of interconnected struts are configured such that the frame is radially compressible to a compressed configuration and radially expandable to a first expanded configuration and a second expanded configuration, the second expanded configuration having a greater diameter than the first expanded configuration;

wherein the plurality of interconnected struts form vertical columns of the frame that each have an inflow end apex and an outflow end apex, junctions connecting the vertical columns circumferentially to each other in a ring, commissure portions extending axially from the some of the junctions toward the outflow end of the frame, and lateral arms that extend circumferentially from outflow ends of the commissure portions to adjacent outflow end apices of the vertical columns.

Example 77. The frame of any example herein, particularly example 76, wherein the frame comprises two of the lateral arms for each of the commissure portions.

Example 78. The frame of any example herein, particularly any one of examples 76-77, wherein when the frame is in the first expanded configuration, the commissure portions are offset radially outwardly from the junctions and the vertical columns, and the lateral arms extend both circumferentially and radially inwardly from outflow ends of the commissure portions to the adjacent outflow end apices of the vertical columns.

Example 79. The frame of any example herein, particularly any one of examples 76-78, wherein the lateral arms pull the commissure portions radially inwardly, relative to the outflow end apices, as the outflow end apices move apart from each other when the frame radially expands from the first expanded configuration to the second expanded configuration.

Example 80. The frame of any example herein, particularly example 79, wherein when the frame is in the second expanded configuration, the commissure portions are radially even with the outflow end apices and the lateral arms extend horizontally between the outflow ends of the commissure portions and the outflow end apices.

Example 81. The fame of any example herein, particularly any one of examples 76-80, wherein the commissure portions comprise a leaflet attachment portion comprising an opening defined between two axial struts of the commissure portions.

Example 82. The frame of any example herein, particularly any one of examples 76-81, wherein the commissure portions comprise an axially extending leaflet attachment portion and an offsetting portion that extends from the respective junction to the leaflet attachment portion, wherein when the frame is in the first expanded configuration, the offsetting portion extends radially outwardly from the respective junction to the leaflet attachment portion, and wherein then the frame is in the second expanded configuration, the offsetting portion does not extend radially outwardly from the respective junction to the leaflet attachment portion.

Example 83. The frame of any example herein, particularly example 83, wherein when the frame is in the first expanded configuration, the commissure portions are offset radially outwardly between 2 mm and 4 mm.

Example 84. A method comprising sterilizing any of the frames, prosthetic heart valves, or delivery apparatus of any of examples 1-83.

The features described herein with regard to any example can be combined with other features described in any one or more of the other examples, unless otherwise stated. For example, any one or more of the features of one valve frame can be combined with any one or more features of another valve frame. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A frame for a prosthetic heart valve, the frame comprising:
a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis;
wherein the plurality of interconnected struts are configured such that the frame is radially compressible to a compressed configuration and radially expandable to one or more expanded configurations; and
commissure portions that are offset radially outwardly relative to the plurality of interconnected struts.

2. The fame of claim 1, wherein the commissure portions are coupled to the plurality of interconnected struts at an inflow end of the commissure portions.

3. The frame of claim 1, wherein the commissure portions comprise a leaflet attachment portion comprising an opening defined between two axial struts of the commissure portions.

4. The frame of claim 1, wherein the commissure portions comprise an axially extending leaflet attachment portion and an offsetting portion that extends radially outwardly from the plurality of interconnected struts to the leaflet attachment portion, wherein the offsetting portion extends both radially outwardly and axially toward the outflow end from the plurality of interconnected struts to the leaflet attachment portion.

5. The frame of claim 1, wherein the plurality of interconnected struts form six diamond-shaped cells that extend between the inflow end of the frame and the outflow end of the frame, wherein the six diamond-shaped cells are connected laterally by six mid-frame junctions positioned between the cells, and the commissure portions extend radially outwardly from three of the six mid-frame junctions.

6. The frame of claim 1, wherein the commissure portions are coupled to the plurality of interconnected struts at one axial end of the commissure portions and free from the plurality of interconnected struts at an opposite, free axial end of the commissure portions and at lateral sides of the commissure portions, such that the commissure portions are cantilevered.

7. The frame of claim 6, wherein the commissure portions terminate at the free axial ends, and the free axial ends are short of the outflow end of the frame, such that the free axial ends of the commissure portions are positioned axially between the inflow end of the frame and the outflow end of the frame.

8. The frame of claim 1, further comprising lateral arms that extend circumferentially from axial ends of the commissure portions to adjacent outflow end apices of the frame;
wherein the frame comprises two of the lateral arms for each of the commissure portions;
wherein when the frame is in a partially radially expanded state, the lateral arms extend both circumferentially and radially inwardly from axial ends of the commissure portions to adjacent outflow end apices of the frame; and
wherein the lateral arms pull the commissure portions radially inwardly, relative to the outflow end apices, as the outflow end apices move apart from each other when the frame radially expands.

9. A prosthetic heart valve comprising:
a frame comprising:
a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis; and
commissure portions that are offset radially outwardly relative to the plurality of interconnected struts; and
a leaflet valve structure positioned within the frame and secured to the commissure portions of the frame;
wherein the prosthetic heart valve is radially compressible to a compressed configuration and radially expandable to one or more expanded configurations.

10. The prosthetic heart valve of claim 9, wherein commissures of the leaflet valve structure are positioned radially outwardly relative to the a plurality of interconnected struts; and wherein the leaflet valve structure articulates about articulation axes that are located at a radial dimension that is equal to or greater than a radial dimension of the plurality of interconnected struts.

11. The prosthetic heart valve of claim 9, wherein the commissure portions comprise an axially extending leaflet attachment portion and an offsetting portion that extends radially outwardly from the plurality of interconnected struts to the leaflet attachment portion, wherein the offsetting portion extends both radially outwardly and axially toward the outflow end from the plurality of interconnected struts to the leaflet attachment portion.

12. The prosthetic heart valve of claim 9, wherein the commissure portions define a maximum radial extent of the frame.

13. The prosthetic heart valve of claim 9, wherein the commissure portions are coupled to the plurality of interconnected struts at one axial end of the commissure portions and free from the plurality of interconnected struts at an opposite, free axial end of the commissure portions and at lateral sides of the commissure portions, such that the commissure portions are cantilevered; and wherein the commissure portions terminate at the free axial ends, and the free axial ends are short of the outflow end of the frame, such that the free axial ends of the commissure portions are positioned axially between the inflow end of the frame and the outflow end of the frame.

14. The prosthetic heart valve of claim 9, further comprising lateral arms that extend circumferentially from axial ends of the commissure portions to adjacent outflow end apices of the frame;
wherein the frame comprises two of the lateral arms extending from each of the commissure portions;
wherein when the frame is in a partially radially expanded state, the lateral arms extend both circumferentially and radially inwardly from axial ends of the commissure portions to adjacent outflow end apices of the frame;
wherein the lateral arms pull the commissure portions radially inwardly, relative to the outflow end apices, as the outflow end apices move apart from each other when the prosthetic heart valve radially expands; and
wherein when the prosthetic heart valve is radially expanded to maximum extent, the commissure portions of the frame are radially even with the outflow end apices of the frame, and the lateral arms extend horizontally between the axial ends of the commissure portions and the outflow end apices.

15. The prosthetic heart valve of claim 9, wherein when the prosthetic heart valve is in an expanded configuration, the commissure portions of the frame are configured to flex radially inwardly relative to the plurality of interconnected struts when the leaflet valve structure closes during diastole to help the leaflet valve structure fully coapt and prevent retrograde flow.

16. A delivery apparatus comprising:
a handle portion;
a shaft portion extending distally from the handle portion; and
a prosthetic heart valve mounted at a distal portion of the shaft portion in a radially compressed configuration;
wherein the prosthetic heart valve comprises an annular frame and a valve structure within the frame, and is radially expandable to one or more expanded configurations;
wherein the frame comprises:
a plurality of interconnected struts forming an annular shape extending circumferentially around a central flow axis, the frame having an inflow end facing one axial direction and an outflow end facing an opposite axial direction, and the frame having a radially inner side facing inwardly toward the central flow axis and a radially outer side face away from the central flow axis; and
commissure portions that are offset radially outwardly relative to the plurality of interconnected struts, the commissure portions being secured to the valve structure; and
wherein the delivery apparatus is configured to deliver the prosthetic heart valve to an implant location within a heart and to cause or allow the prosthetic heart valve to radially expand to the one or more expanded configurations at the implant location.

17. The delivery apparatus of claim 16, wherein the frame comprises shape memory material and the prosthetic heart valve is self-expandable, and wherein the shaft portion comprises a sheath that holds the prosthetic heart valve in the radially compressed configuration and the handle comprises an actuator that causes the sheath to move axially to uncover the prosthetic heart valve and allow the prosthetic heart valve to self-expand.

18. The delivery apparatus of claim 16, wherein the shaft portion comprises an inflatable balloon positioned within the radially compressed prosthetic heart valve and the delivery apparatus is configured to inflate the balloon to radially expand the prosthetic heart valve.

19. The delivery apparatus of claim 16, wherein the frame further comprises axially oriented screws coupled to the plurality of interconnected struts, and rotation of the screws by the delivery apparatus causes the frame to axially shorten and causes the prosthetic heart valve to radially expand; and
wherein the shaft portion comprises at least one actuator coupled to the frame of the prosthetic heart valve and the handle comprises a controller that causes the at least one actuator to turn at least one of the axially oriented screws of the frame of the prosthetic heart valve to cause the prosthetic heart valve to radially expand.

20. A method comprising using the delivery apparatus of claim 16 to deliver the prosthetic heart valve to the implant location within the heart and to implant the prosthetic heart valve at the implant location.
